(19) United States
(12) Patent Application Publication
(10) Pub. No.: US 2005/0010122 A1
(43) Pub. Date: Jan. 13, 2005

(54) Spatial heterogeneity of repolarization waveform amplitude to assess risk of sudden cardiac death

(75) Inventors: Bruce D. Nearing, North Reading, MA (US); Richard L. Verrier, Wellesley Hills, MA (US)

Correspondence Address: STERNE, KESSLER, GOLDSTEIN & FOX PLLC, 1100 NEW YORK AVENUE, N.W., WASHINGTON, DC, 20005, US

(73) Assignee: Beth Israel Deaconess Medical Center, Boston, MA (US)

(21) Appl. No.: 10/846,845

(22) Filed: May 17, 2004

Related U.S. Application Data
(60) Provisional application No. 60/470,440, filed on May 15, 2003.

(51) Int. Cl. A61B 5/0402

(52) U.S. Cl. 600/509

(57) ABSTRACT

Exercise-induced T-wave alternans (TWA) in coronary artery disease patients reflects significant levels of spatial heterogeneity of repolarization, which may underlie the predictive utility of TWA in estimating risk of sudden cardiac death. A method for assessing spatial heterogeneity of repolarization of a heart of a patient includes the following steps: simultaneously sensing an ECG signal from each of a plurality of spatially separated leads attached to the patient; for a plurality of N beats in each of the ECG signals, identifying a JT interval of each beat; and for corresponding ones of the JT intervals of the ECG signals, calculating a second central moment indicative of spatial heterogeneity of repolarization.

### CROSS REFERENCE TO RELATED APPLICATIONS

This application claims priority under 35 U.S.C. §119(e) to commonly owned, co-pending U.S. Provisional Patent Appl. No. 60/470,440, filed May 15, 2003.

### STATEMENT REGARDING FEDERALLY-SPONSORED RESEARCH AND DEVELOPMENT

Part of the work performed during development of this invention utilized U.S. Government funds. The U.S. Government has certain rights in this invention.

### BACKGROUND OF THE INVENTION

1. Field of the Invention

The present invention relates to the field of cardiology. More specifically, the invention relates to non-invasive identification and management of individuals at risk for sudden cardiac death. Cardiac vulnerability to ventricular fibrillation, the mode of sudden death, is tracked by analysis of an electrocardiogram.

2. Related Art

A sizeable body of evidence demonstrates that T-wave alternans (TWA), a beat-to-beat fluctuation in the T-wave of an electrocardiogram (ECG) indicating electrical instability of the myocardium, provides a useful means for arrhythmia risk stratification. However, surprisingly little is known about the electrophysiologic basis for TWA during exercise stress testing in patients with stable coronary artery disease, a widespread, relatively low-risk patient population of 13.5 million in the United States alone. Also limited is the available body of information on TWA levels in normal subjects in the coronary prone age.

Fluctuations in T-wave morphology particularly in the form of TWA have been linked to increased susceptibility to ventricular fibrillation (VF). Numerous experimental studies have demonstrated that the magnitude of TWA can gauge vulnerability to VF under diverse physiologic and pharmacologic interventions. Clinically, TWA has also proved promising in assessing risk for ventricular arrhythmias in patients with ischemic heart disease, heart failure, dilated cardiomyopathy, long QT syndrome, acute myocardial infarction, and other conditions. For a detailed discussion of T-wave alternans, see U.S. Pat. No. 5,921,940 to Verrier and Nearing, which is incorporated herein by reference in its entirety as if reproduced in full below.

Heterogeneity of repolarization is an electrophysiologic mechanism commonly linked to arrhythmogenesis and increasingly implicated in TWA. What is needed is a method and apparatus for quantifying and tracking heterogeneity of repolarization.

### SUMMARY OF THE INVENTION

Exercise-induced TWA in coronary artery disease patients reflects significant levels of T-wave heterogeneity (TWH) (spatial heterogeneity of repolarization), which may underlie the predictive utility of TWA in estimating risk of sudden cardiac death. TWA and TWH provide complementary means to assess cardiac electrical instability. Essentially, TWA is a measure of temporal inhomogeneities monitored from a single lead. TWH provides a spatial measure of heterogeneity, as signals from multiple leads are compared on a beat-by-beat basis. There is a complementary benefit that related physiologic phenomena are evaluated. Because these measures rely on different principles of determination, the potential for artifact in one may be offset by measurement principles of the other. For example, respiratory and rhythmic motion artifacts can disrupt the measurement of TWA, but, because TWH is a spatial approach, it is not disrupted by these potential artifacts.

The invention includes a method and apparatus for assessing spatial heterogeneity of repolarization of a heart of a patient. The method includes the steps of: simultaneously sensing an ECG signal from each of a plurality of spatially separated leads attached to the patient; for a plurality of N beats in each of the ECG signals, identifying a JT interval of each beat; and for corresponding ones of the JT intervals of the ECG signals, calculating a second central moment indicative of spatial heterogeneity of repolarization.

The invention also includes a method for identification and screening risk of sudden cardiac death based on TWH. A TWH measure is taken for a patient. The TWH measure is then scaled based on a desired R-wave amplitude. After scaling, the scaled value can be compared to a normative value.

Further embodiments, features, and advantages of the present invention, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the relevant art to make and use the invention.

FIG. 1 is a flow chart illustrating an embodiment of the invention for calculating a measure of T-wave heterogeneity.

FIG. 2 shows superimposition of four simultaneous simulated ECG waveforms (A-D) and illustrates heterogeneity of T-wave morphology.

FIG. 3 illustrates the results of testing second central moment analysis of T-wave heterogeneity (TWH) for accuracy in simulated ECGs in which TWH was elevated in 50 equal intervals from zero to 800 μV. Sample simulated ECGs with their TWH values are also shown.

FIG. 4 illustrates the results of testing second central moment analysis of T-wave heterogeneity (TWH) for accuracy in simulated ECGs with ST-segment deflections of zero to 1000 μV. The output TWH value remained equal to the input TWH value of 99.6 μV. Sample simulated ECGs with their ST-segment deviations and calculated TWH values are also shown.

FIG. 5 shows simulated ECGs with U waves of increasing amplitude from zero to 500 μV and shows how the output TWH value remained equal to the input TWH value of 99.6 μV. Sample simulated ECGs with U waves and calculated TWH values are also shown.

FIG. 6 shows simulated ECGs with T-wave inflections of increasing amplitude from zero to 500 μV and shows how the output TWH value remained equal to the input TWH value of 99.6 μV. Sample simulated ECGs with T-wave inflections and TWH values are also shown.

FIG. 7 is a graph showing TWH versus time, in which second central moment analysis revealed increased TWH after the start of occlusion in canines in which myocardial ischemia provoked VF versus those without VF.

FIG. 8 depicts TWH in 4-electrode epicardial plaque electrograms at 4 min of occlusion in a representative canine in which myocardial ischemia provoked VF (right panel) and did not provoke VF in a second representative canine (left panel). Superimposition (lower panels) provides visual evidence of the significant differences in repolarization patterns.

FIG. 9 shows representative ECGs from a dog that exhibited VF. Note the progression of T-wave complexity in electrograms monitored from a 4-electrode plaque preceding VF.

FIG. 10 shows summary TWH data for twelve dogs.

FIG. 11 shows representative precordial ECGs recorded from two closed-chest pigs before and during angioplasty-balloon induced LAD coronary artery occlusion, one with and one without myocardial ischemia-induced VF.

FIG. 12 shows superimposition of the precordial ECGs of FIG. 11.

FIG. 13 shows differing amplitudes of precordial TWH in seven pigs in which VF was provoked by LAD coronary artery occlusion versus five pigs in which VF was not provoked.

FIG. 14 shows that TWA results of exercise treadmill testing (ETT) for normal and CAD patients.

FIG. 15 shows TWH results of ETT for normal and CAD patients.

FIG. 16 shows concurrent ECGs that exhibit ETT-induced increase in TWA and TWH in a patient with coronary artery disease.

FIG. 17 is a flowchart showing a method for identifying and screening risk of sudden cardiac death based on TWH.

The present invention will be described with reference to the accompanying drawings. The drawing in which an element first appears is typically indicated by the leftmost digit(s) in the corresponding reference number.

### DETAILED DESCRIPTION OF THE INVENTION

While specific configurations and arrangements of the invention are discussed herein, it should be understood that this is done for illustrative purposes only. A person skilled in the relevant art will recognize that other configurations and arrangements can be used without departing from the spirit and scope of the present invention.

Just prior to onset of ischemia-induced VF, there is a progressive increase in the complexity of T-wave oscillations that heralds the onset of the arrhythmia. The oscillations escalate from the ABAB pattern of TWA to tripling (ABCABC) or quadrupling (ABCDABCD) patterns that lead abruptly to more complex oscillatory T-wave forms and VF. Episodes of discordant TWA, with alternation out of phase in neighboring epicardial sites, frequently antecede VF.

Based on this finding, the inventors postulated that complex T-wave oscillations and discordant TWA reflect states of heightened spatial heterogeneity of repolarization. To test this hypothesis, the inventors developed a second-central-moment analysis technique to quantitate heterogeneity of T-wave morphology from multiple epicardial or precordial leads. This approach has the intrinsic advantages that heterogeneity throughout the entire T-wave is assessed and that the measurement is not unduly weighted by protracted termination or inflections in the T wave, biphasic forms, ST-segment changes, or the presence of U waves, features that limit accurate measurement by conventional QT-interval analysis. The linkage between spatial T wave heterogeneity (TWH) and onset of VF was tested in open- and closed-chest experimental preparations and in a clinical study using epicardial electrograms and/or precordial leads.

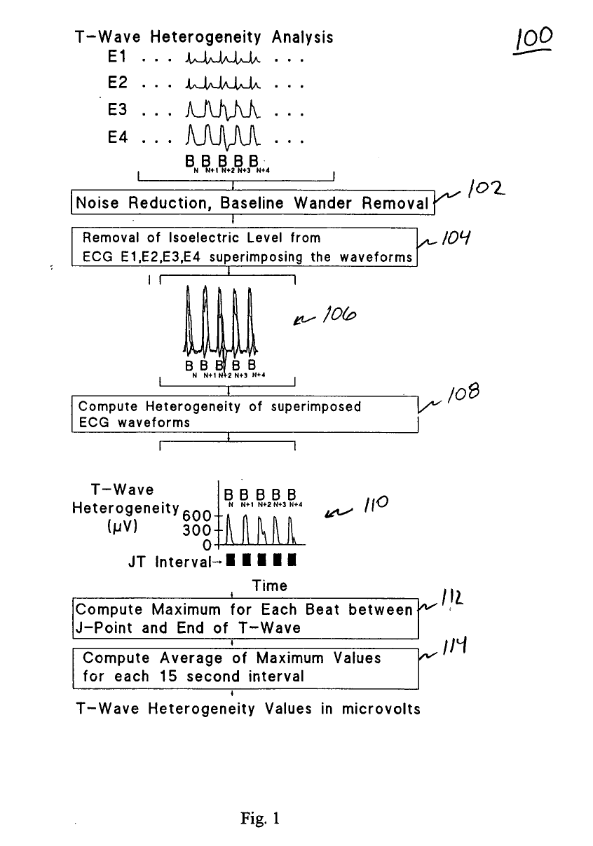

This method of the invention is illustrated in FIG. 1. In a representative experiment in which coronary artery occlusion subsequently resulted in VF, four ECGs (E1-E4) were simultaneously obtained from four electrodes of a plaque situated in the anticipated zone of myocardial ischemia.

In step 102, the ECGs were filtered to reduce high-frequency noise and to remove baseline wander. Ventricular and supraventricular premature beats as well as beats with a high noise level were removed. In step 104, the isoelectric level was made uniform for each electrode.

In step 104, the ECG waveforms (i.e., the successive beats Bn, Bn+1, Bn+2, etc.) are also superimposed on one another and displayed to an operator for visual inspection. The resulting superimposed waveforms are illustrated at 106. The superimposed waveforms are useful for an operator to visually confirm the presence of TWH. It should be noted, however, that this is an optional step. Superimposition and display are not required for computation of a TWH measure.

In step 108, the ECG waveforms were analyzed to compute spatial heterogeneity for each ECG waveform. The invention focuses primarily on analysis of spatial heterogeneity of repolarization. Repolarization is represented by the T wave portion of an ECG signal. Therefore, the invention focuses on analysis of the T wave portion of ECG signals. The T wave can be defined, for example, as the portion of the ECG from the J point to the end of (or some arbitrary point on) the T wave. This may also be called the “JT interval.” The J point is the point in the ECG signal where the signal returns to the isoelectric value after the S wave. For purposes of the invention, the term “JT interval” means at least a portion of the part of the ECG that is representative of repolarization.

In one embodiment, spatial heterogeneity is computed using second central moment analysis. The rationale for calculating heterogeneity of repolarization by analyzing and comparing the second central moment of simultaneous T waves in local electrograms of several precordial leads is to measure spatial variation in morphology over the entire T wave. Second central moment is a concept from Newtonian mechanics that refers to a measure of splay in area (measured in microvolts squared) around the first moment, i.e., the average amplitude of the entire T wave, as its axis. Specifically, the square root of the second central moment of simultaneous T-waves is computed to measure the deviation of T-wave morphology about the mean value.

Second central moment is a square function, since it is the computation of area around a central axis. In this representative experiment, the square root of the second central moment of the four simultaneous T waves (from the J point to the end of the T wave) was computed (step 108) from the waveforms to measure deviation among the T waves. A measure of T-wave heterogeneity for the ECG signals is illustrated at 110 for beats B_{N}-B_{N+4}. Note that this is a continuous function representing TWH on a beat-by-beat basis.

This analytical approach avoids the confounding influences of inaccurate identification of the terminal portion of the T wave, which may be obscured by ST-segment changes, the presence of U waves, or T-wave inflections. Measurement of TWH by second central moment analysis differs from previous applications of root-mean-square calculations that have been employed for identifying the end of the T wave.

In one embodiment, the analytical approach of step 108 is as follows.

R waves are identified and an average waveform is computed on a point-by-point basis using the following equation:

$$\overline{e ( t )} = \frac{1}{4} {\sum }_{i = 1}^{4} {e}_{i} ( t )$$

wherein e(t) represents the time varying ECG signal, and i represents the specific ECG signal.

The second central moment of the T-wave is then calculated by taking the root-mean-square deviation of the JT interval, which occurs, for example, from about 60 to about 290 msec after the R wave:

$$σ ( t ) = \sqrt{\frac{1}{4} {\sum }_{i = 1}^{4} {( {e}_{i} ( t ) - \overline{e ( t )} )}^{2}}$$

The time varying result of this calculation is depicted at 110 in FIG. 1.

At step 112, the maximum TWH value is calculated on a beat-to-beat basis using the following equation:

$$TWH = {σ}_{max} = \begin{matrix}MAX \\ t\end{matrix} ( t )$$

As an alternative to computing the maximum TWH value for each beat, the time-varying TWH value for each beat can be averaged to produce an average value for each beat.

At step 114, the results of step 112 (whether max or average values) are averaged for a predetermined time interval (e.g., 15 seconds) to produce a T-wave heterogeneity value in microvolts.

The accuracy of this method was examined by measuring T-wave heterogeneity (TWH) values in simulated ECGs generated by a C++ program, having P waves, R waves, T waves, and ST-segments approximated by geometric shapes whose relative timing and amplitude were similar to surface ECGs. The resulting TWH readings were compared with input TWH of zero to 800 μV that was centered during the first half of the T wave, the period in the cardiac cycle when enhanced heterogeneity of repolarization is known to occur. Detection of TWH of this level should be sufficient for most analyses. The method's capacity to measure TWH simulated in ECGs with ST-segment deviation, prominent U waves, and T-wave inflections, which are common in routine clinical and experimental ECGs and which confound current approaches to measuring heterogeneity of repolarization, was also assessed.

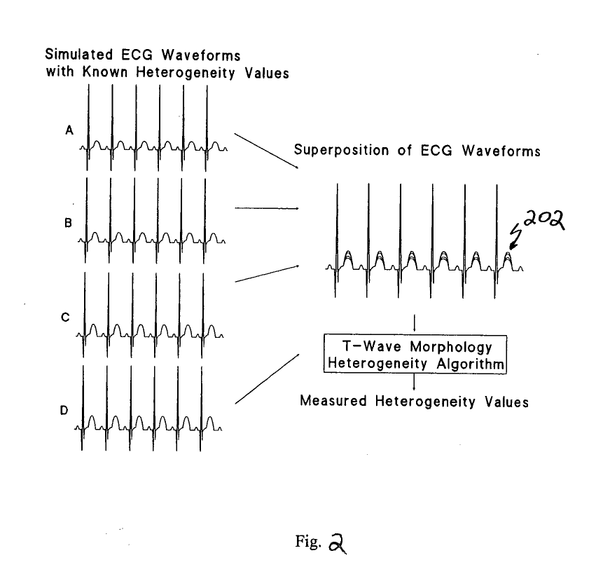

The method accurately tracked inhomogeneities in T-wave morphology, which are visible when the waveforms are superimposed. This is illustrated in FIG. 2, which shows superimposition of four simultaneous simulated ECG waveforms (A-D). Note, as indicated by reference numeral 202, the heterogeneity of T-wave morphology, the parameter measured by second central moment analysis.

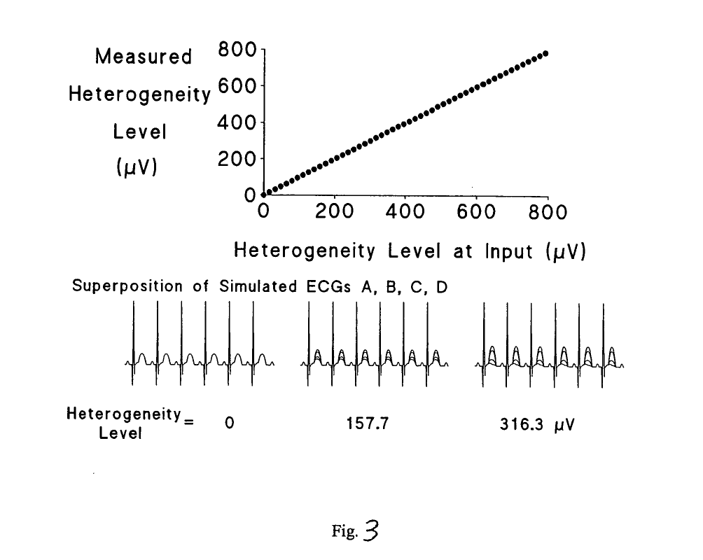

Second central moment analysis of T-wave heterogeneity (TWH) was tested for accuracy in simulated ECGs in which TWH was elevated in 50 equal intervals from zero to 800 μV. Regression analysis yielded a correlation of r²=0.999, with p<0.001. Sample simulated ECGs with their TWH values are shown in FIG. 3. Note the linear relationship with a correlation coefficient of r²=0.999 (p<0.001) that can be observed between the TWH value estimated by second central moment analysis and the input value in the simulated ECGs. FIG. 3 also shows superimposition of ECGs A-D with heterogeneity levels of 0.0, 157.7 and 316.3 μV.

Note that, in this simulation, the results were not affected by the introduction of ST-segment deviation, U waves, and T-wave inflections, which may obscure the terminal portion of the T wave. The simulated ECGs contained a constant TWH of 99.6 μV, and the measured TWH differed less than one percent (<1%) from the new waveforms.

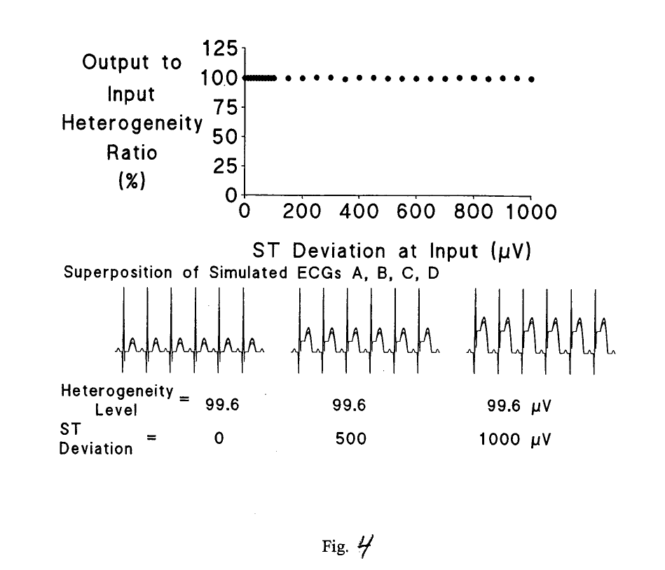

For example, FIG. 4 shows the effects of ST-segment deviation on the TWH measure. Simulated ECGs with ST-segment deflections of zero to 1000 μV were tested. As illustrated, second central moment calculation of TWH is not affected by ST-segment deviation. The output TWH value remained equal to the input TWH value of 99.6 μV. FIG. 4 also shows superimposition of ECGs A-D with ST deviation levels of 0.0, 500 and 1000 μV. Note that the calculated TWH value (99.6 μV) is the same for all three ST deviation levels.

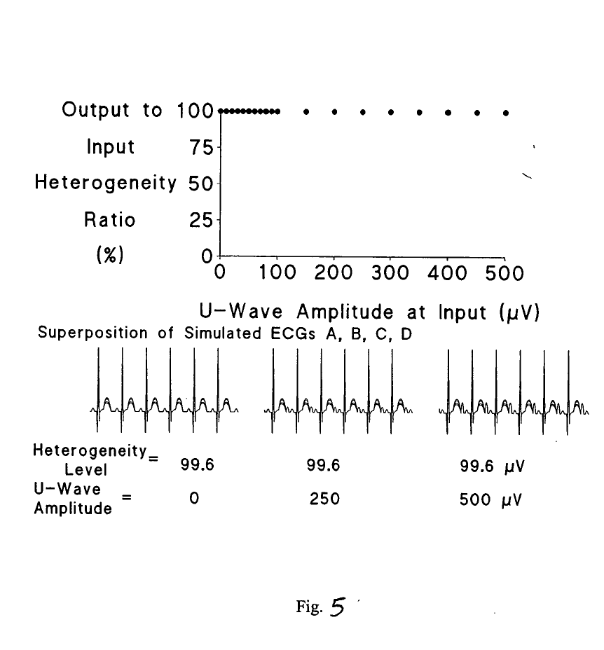

FIG. 5 shows the effects of U waves on the TWH measure. Simulated ECGs with U waves of increasing amplitude from zero to 500 μV were tested. As illustrated, second central moment calculation of TWH is not affected by the occurrence of U waves, which obscure the terminal portion of the T-wave. The output TWH value remained equal to the input TWH value of 99.6 μV. Sample simulated ECGs with U waves and calculated TWH values are also shown. FIG. 5 also shows superimposition of ECGs A-D with U-wave amplitudes of 0.0, 250 and 500 μV. Note that the calculated TWH value (99.6 μV) is the same for all three U-wave amplitudes.

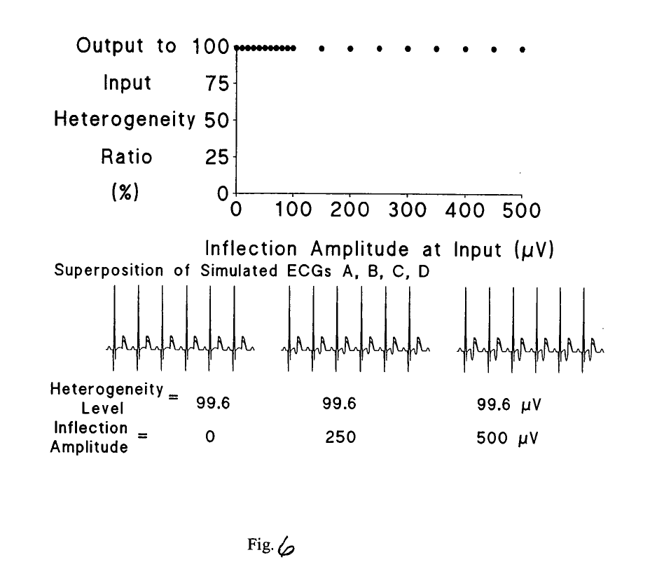

FIG. 6 shows the effects of T-wave inflections on the TWH measure. Simulated ECGs with T-wave inflections of increasing amplitude from zero to 500 μV were tested. As illustrated, second central moment calculation of T-wave heterogeneity (TWH) is not affected by T-wave inflections. The output TWH value remained equal to the input TWH value of 99.6 μV. FIG. 6 also shows superimposition of ECGs A-D with T-wave inflection amplitudes of 0.0, 250 and 500 μV. Note that the calculated TWH value (99.6 μV) is the same for all three inflection amplitudes.

Referring back to FIG. 1, ECG waveforms E1-E4 are used to illustrate operation of the invention. The ECG signals for use with the present invention may be sensed and processed, for example, as described in U.S. Pat. No. 5,921,940. For example, in one embodiment, the ECG signals are sensed from precordial ECG leads using a conventional ECG machine. Each sensed ECG signal contains a plurality N of R-R intervals. For spatial heterogeneity analysis, a plurality of ECG signals (i.e., ECG signals sensed from a plurality of spatially different sites) is required. Any number of ECG signals greater than two can be used. For example, all six precordial leads can be used. It may also be possible to use the limb leads and/or augmented leads in lieu of, or in addition to, the precordial leads.

After sensing, each ECG signal is high-pass filtered and amplified. The amplified ECG signals are then low-pass filtered to limit the signal bandwidth before they are digitally sampled. Once sampled, the digitized data may then be stored on a storage device for analysis or may be analyzed in real time. This filtering and processing is illustrated by step 102 in FIG. 1.

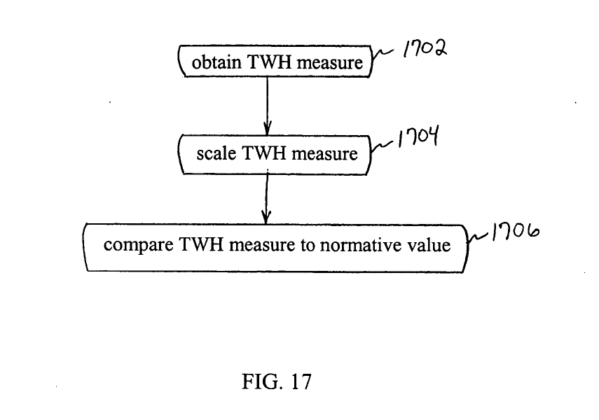

The invention also includes a method for identification and screening risk of sudden cardiac death based on TWH. This method is illustrated in the flow chart of FIG. 17. As shown, a TWH measure is taken for a patient in a step 1702. The TWH measure is taken, for example, using the method of FIG. 1 discussed above. In a step 1704, the TVH measure is scaled based on a desired R-wave amplitude. Finally, in a step 1706. the scaled TWH measure is compared to a normative value.

This process of TWH scaling, permits inter-individual comparison of TWH values. For example, in some individuals, a sensed ECG signal may have a reduced amplitude due to poor electrode contact. In other individuals, a sensed ECG signal may have reduced amplitude because the heart is diseased. Scaling permits normalization of the TWH values for comparison to one another and for comparison to predetermined values (i.e., normative values or thresholds). In the embodiment of FIG. 17, R-wave amplitude is used as a reference. That is, the R-wave amplitude of a selected ECG is compared to a normative R-wave amplitude value. If the selected ECG has an R-wave amplitude that is less than the normative R-wave amplitude, then the TWH measure calculated for the selected ECG signal is multiplied by the ratio (i.e., normative R-wave amplitude divided by selected R-wave amplitude) to scale the TWH measure. For example, if the selected ECG has an R-wave amplitude that is 0.7 times that of the normative R-wave amplitude, then the TWH measure of the selected ECG signal is multiplied by the ratio 10/7 to scale the TWH measure.

Alternatively, the selected ECG signal may be scaled prior to calculation of the TWH measure. For example, if the selected ECG signal has an R-wave amplitude that is less than the normative R-wave amplitude, then the selected ECG signal is multiplied by the ratio (i.e., normative R-wave amplitude divided by selected R-wave amplitude) to scale the selected ECG signal. Thereafter, the TWH measure can be calculated for the patient.

After scaling, the TWH measure from the patient can be properly compared to threshold data, averaged data, or historical data. For historical data, scaling may facilitate historical comparison of TWH measures from a patient over time as a disease changes ECG amplitude.

The present invention can be implemented in computer software, hardware, or firmware. For example, the invention may be implemented in a conventional ECG machine, pacer, implantable cardioverter defibrillator (ICD), Holter monitor, heart monitoring unit, or using dedicated hardware. For a discussion of such hardware, see the above-referenced U.S. Pat. No. 5,921,940.

Animal Experiments

Animal experiments were conducted under a surgical plane of anesthesia according to protocols approved by the institutional animal care and use committee and standards set by the National Institutes of Health and described in American Physiological Society's Guiding Principles in the Care and Use of Animals.

Open-Chest Canine Study

The epicardial electrograms from 12 mongrel dogs of either sex, weighing from 18 to 26 kg, were derived from a recent study in which the first demonstration of complex oscillatory T-wave forms leading to VF was reported. See, Nearing BD, Verrier RL, “Progressive increases in complexity of T-wave oscillations herald ischemia-induced VF,” Circulation Research, 2002, vol. 91, pp. 727-732 (hereafter, “Nearing and Verrier 2002”). The focus of the canine study was to provide a completely de novo analysis of spatial heterogeneity of T-wave morphology concurrent with the onset of complex T-wave oscillations. Canines of either sex were preanesthetized with xylazine (0.24 mg/kg, s.c.) and anesthetized with alpha-chloralose (150 mg/kg, i.v., with supplemental doses of 600 mg in 60 ml saline as required). Following thoracotomy, the left anterior descending (LAD) coronary artery was occluded to induce myocardial ischemia. Spatial TWH was analyzed from ECGs obtained from 4 Ag—AgCl electrodes of 1-mm diameter spaced at 45°, 135°, 225°, and 315° around a 5-mm circular Plexiglas plaque, which was placed on the epicardium in the expected zone of myocardial ischemia and sutured away from the electrodes to avoid current of injury. Bipolar ECGs were obtained with each of the four epicardial plaque electrodes as the negative poles and a needle electrode placed transcutaneously in the lower left hip region as the common positive reference pole. Heart rate was maintained constant by right atrial pacing at 150 beats/min.

The effects of myocardial ischemia were evaluated by comparing baseline TWH at 4 min before occlusion with TWH levels monitored during an 8-min period of LAD coronary artery occlusion. T-wave multupling was quantified by complex demodulation by computing the area under the T wave from a series of samples from 60 to 220 ms after the R wave and analyzing the result with complex exponentials at the alternating, tripling, and quadrupling frequencies. See, Nearing & Verrier 2002. Complex oscillatory T-wave forms were considered present when complex demodulation results decreased while repeating T-wave patterns remained visible. Episodes of discordant TWA in the epicardial 4-electrode plaque were identified by multiplying the T-wave areas of all pairs of electrodes for each beat. When discordant TWA was present, the product was negative because the factors were positive and negative.

Closed-Chest Porcine Study

Spatial TWH in precordial ECGs (V2, V3, V4) was studied in 12 closed-chest Yorkshire pigs of either sex, weighing 32.9±1.5 kg (range: 22.9 to 39.5 kg) during right atrial pacing at 120 beats/min. The pigs were preanesthetized with telazol (4.7 mg/kg, i.m.) and xylazine (2.2 mg/kg, i.m.) and anesthetized with alpha-chloralose (bolus, 100 mg/kg, i.v., followed by continuous infusion, 40 mg/kg/hr, i.v.). Myocardial ischemia was induced by intraluminal occlusion of the left anterior descending (LAD) coronary artery with an angioplasty balloon using standard techniques and equipment. Specifically, under fluoroscopic guidance, the left main coronary artery was cannulated with an 8Fr Judkins right guide catheter (JR4 with side holes, Boston Scientific, Natick Mass.). An angioplasty guidewire (0.014″ Wizdom guidewire, Cordis, Hialeah Fla.) was threaded through the LAD coronary artery and past the second diagonal branch. An angioplasty balloon, 2.5- to 3.5-mm in diameter and 10- to 20-mm long (Boston Scientific, Natick Mass.), was passed over the guidewire to position the proximal end just beyond the first diagonal branch and was inflated to occlude the vessel completely, as verified with angiography. This closed-chest model of intracoronary artery occlusion yielded a high incidence of ventricular fibrillation (VF).

Preprocessing of Experimental Laboratory ECGs

Recording and analysis of data were performed with commercial equipment (e.g., a MARS workstation, available from GE Medical Systems Information Technologies, Milwaukee, Wis.). Briefly, ECGs were low-pass filtered at 50 Hz, sampled at 500 Hz per channel, and stored on rewritable optical disks by Streamer software. The data were down-sampled to 125 Hz for analysis on the MARS Workstation. Because the R-wave amplitude of the epicardial ECGs is larger than that of the surface ECG, the epicardial ECGs were scaled down by a factor of ten for analysis. Ectopic beats, ventricular arrhythmias, or artifacts automatically identified by the MARS workstation were verified by a trained operator and removed from the analysis.

ECGs were low-pass filtered to remove high-frequency noise using an 8th order digital Butterworth filter with a comer frequency of 50 Hz. Baseline wander, a low-frequency artifact caused by changes in thoracic impedance during respiration, was estimated based on isoelectric points in each ECG beat by calculating a cubic spline and was subtracted from the ECG signal.

Statistical tests were carried out with an SAS statistical package (SAS Institute, Cary, N.C.). TWH levels were compared by one-way ANOVA with Tukey correction for multiple comparisons. Values=means±SEM, p<0.05.

Results of Open-Chest Canine Study

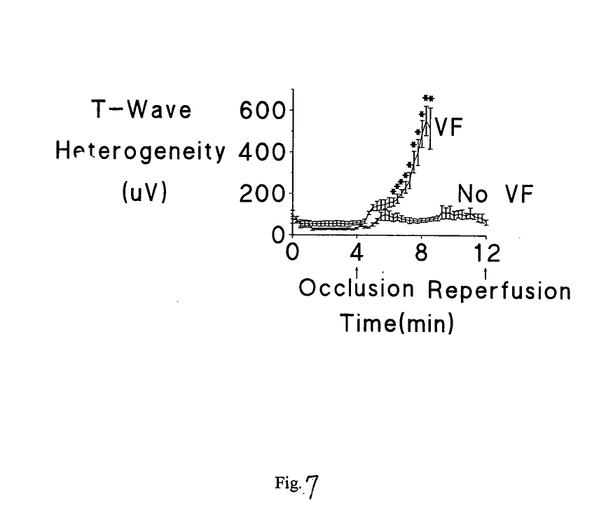

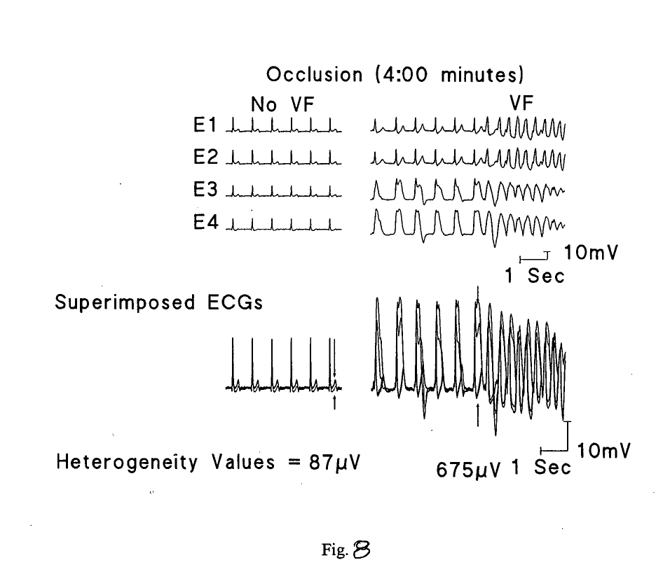

TWH, as continuously measured by second central moment analysis, began to increase significantly at 2.25 min after the start of LAD occlusion and continued to increase in the 6 animals in which myocardial ischemia-induced VF ensued at 4.36±0.14 min. This is illustrated in FIG. 7. TWH levels observed shortly before VF were markedly higher than in the 6 animals without VF at the same time point (563±56 vs 139±36 μV, p<0.01). FIG. 8 shows ECGs and heterogeneity values for a representative animal with VF (right panels) and for a representative animal without VF (left panels). Note that the increase in TWH was not significant in the animals in which VF was not provoked (from 58±6 at preocclusion baseline to 139±36 μV, NS).

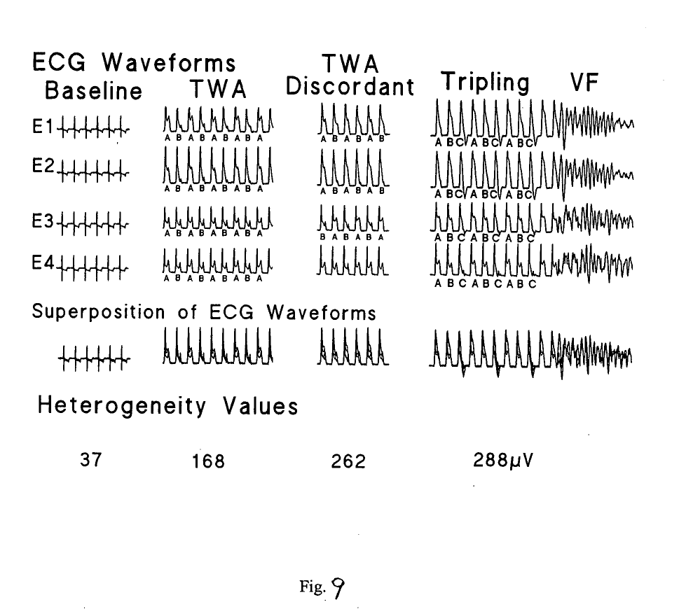

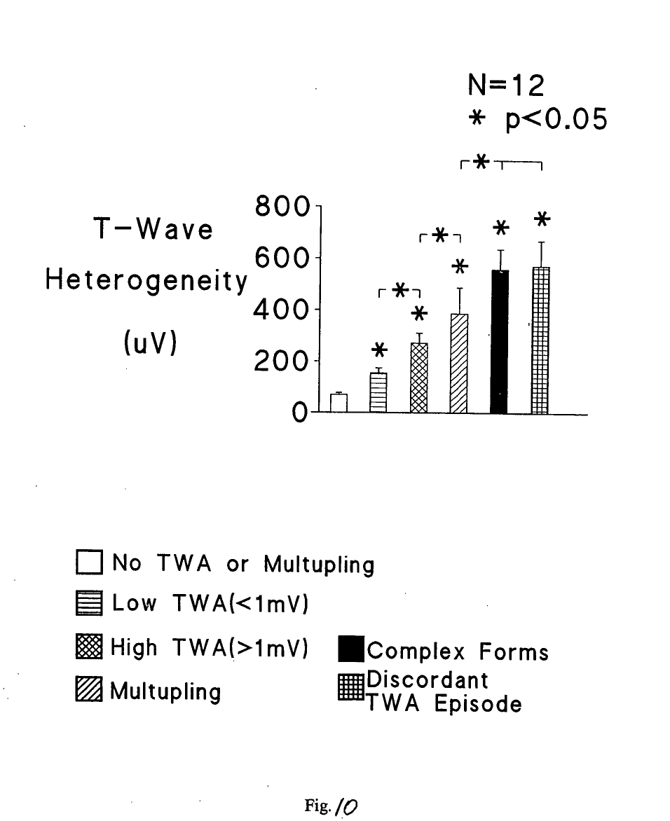

Successive, significant increases in TWH were observed as T-wave oscillations appeared and became more complex. Increasing levels of TWH were concomitant with increased TWA magnitude from preocclusion baseline 70±8 μV to 155±19 μV at low levels of TWA (<1 mV) and to 272±39 μV at higher levels of TWA (>1 mV). The greatest amount of heterogeneity was observed during T-wave tripling and quadrupling (386±100 μV), complex oscillatory T-wave forms (560±76 μV), and episodes of discordant TWA (572±98 μV), features that distinguished animals in which myocardial ischemia provoked VF. FIG. 9 shows a representative example from an animal that exhibited VF. FIG. 10 shows summary data for all twelve dogs. For all comparisons, p<0.05.

Results of Closed-Chest Porcine Study

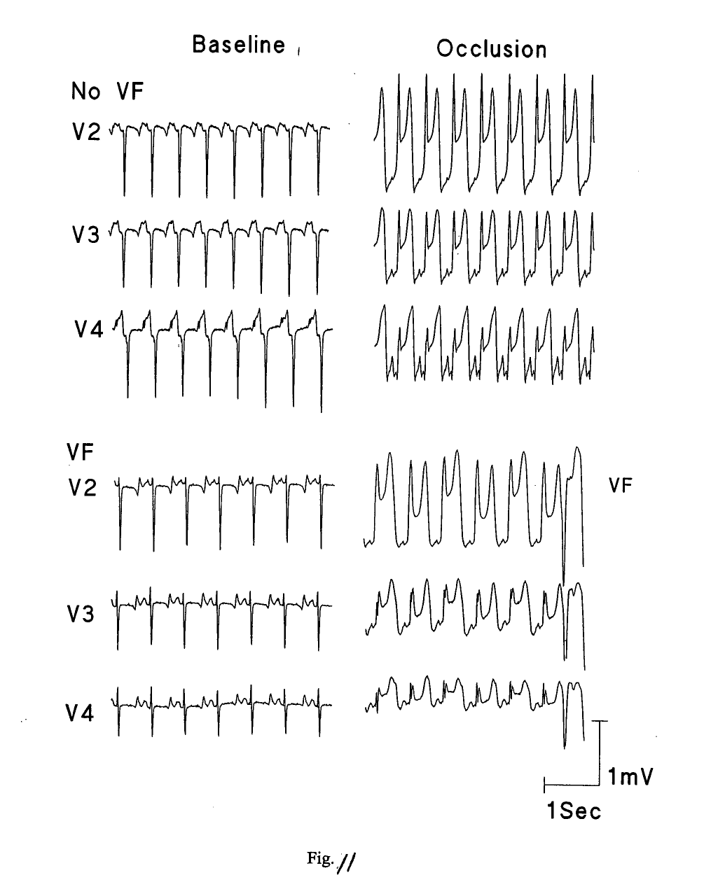

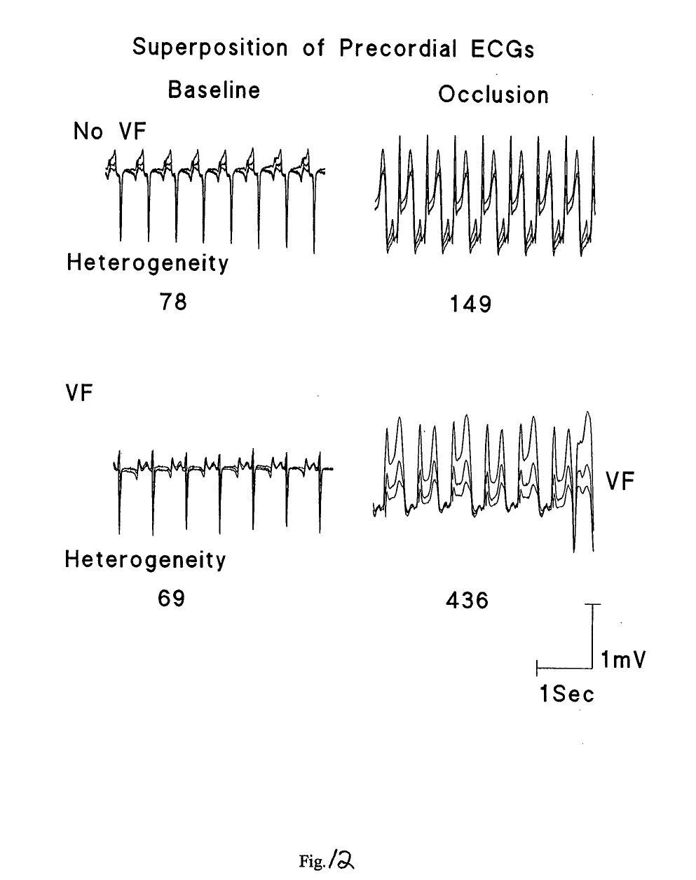

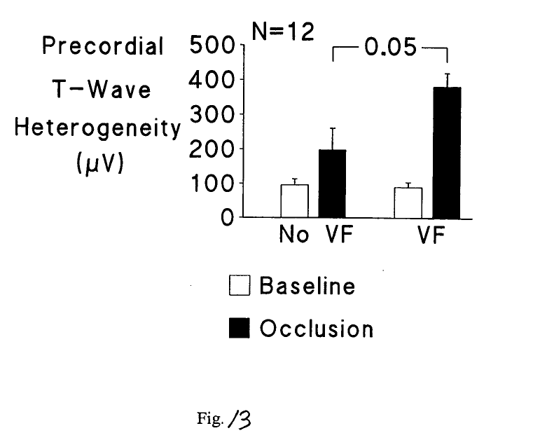

Angioplasty-balloon induced LAD occlusion provoked a significant increase in precordial TWH (from 90±14 at pre-occlusion baseline to 382±39 μV shortly before VF, p<0.05) in the 7 of 12 animals in which myocardial ischemia provoked VF. FIG. 11 shows a representative examples of ECGs from an animal that exhibited VF and from an animal that did not exhibit VF. FIG. 12 shows superimposition of the data of FIG. 11. FIG. 13 shows summary data for all twelve pigs.

Note that neither VF nor increased levels of TWH occurred in five of the pigs (from 96±17 at pre-occlusion baseline to 199±61 μV, NS, at the same time point). Likewise, in the seven pigs that experienced VF, there was a significant rise in TWA in V2, the lead exhibiting the greatest TWA magnitude from 18±2 at baseline to 236±34 μV (p<0.05) at 3.5 min of occlusion, just prior to VF, but the rise in TWA was not significant in the animals that did not experience VF (lead V2: from 14±2 at baseline to 40±13 μV, NS). T-wave multupling was not evident, probably because the resolution of precordial leads is considerably less than that of local epicardial electrograms.

Discussion of Animal Study

The main objective of the animal study was to test the hypothesis that complex oscillations in T-wave morphology culminating in VF during acute myocardial ischemia reflect a state of increased spatial heterogeneity of repolarization. Therefore, TWH were evaluated by measuring the second central moment of simultaneous T waves recorded from several epicardial sites within the ischemic zone or from precordial leads. Increasing levels of TWH indicated the development of increased electrical instability and heralded the onset of myocardial ischemia-induced VF.

Second central moment analysis indicated a significant, progressive increase in TWH in epicardial or precordial leads among animals vulnerable to myocardial ischemia-induced VF, with a close correspondence to the crescendo in complex T-wave oscillations. Specifically, heightened levels of TWH were temporally associated with augmented electrical instability as evidenced by increased TWA magnitude and the onset of T-wave multupling, complex oscillatory T-wave forms, discordant TWA episodes, and finally VF. In hearts in which fibrillation did not occur, no myocardial ischemia-induced increase in TWH was evident and neither T-wave multupling, complex oscillatory T-wave forms, nor discordant TWA ensued. Furthermore, TWH was discovered to track the myocardial ischemia-induced increase in electrical instability established by VF threshold testing studies and the incidence of myocardial ischemia-induced ventricular tachyarrhythmias during the first 4 to 5 min of occlusion of a coronary artery. Heart rate was not a factor in the rise in TWH as it was held constant by right atrial pacing.

The present findings of a close temporal relationship between increased TWH, as measured by the second central moment, and TWA during severe myocardial ischemia are consistent with previous studies employing indirect measures of heterogeneity of repolarization.

The present findings represent the first measurement of TWH concurrent with T-wave multupling. The progressive increase in TWH concomitant with an increase in TWA magnitude and complexity of T-wave oscillations supports the proposition that heightened levels of heterogeneity of repolarization underlie T-wave multupling during the development of VF.

It does not appear likely that conduction block played a role in myocardial ischemia-induced electrical instability evidenced by increased TWH and TWA in these studies. This assumption is based on the fact that these electrophysiologic alterations occurred during 3 to 4 min after onset of coronary artery occlusion. It is well established that conduction abnormalities require a longer period of time to develop.

The ionic bases for the marked rise in spatial TWH in association with multupling remain unidentified. Several lines of evidence implicate the involvement of calcium both in heterogeneity of repolarization and in myocardial ischemia-induced T-wave oscillations. Fluctuations in this ion have been observed in synchrony with repolarization alternans during myocardial ischemia.

Human Clinical Study

The human clinical study was performed in 16 consecutive patients with stable CAD selected from the Vascular Basis for the Treatment of Myocardial Ischemia Study. Exercise ECGs from 16 normal volunteers were also analyzed. Patients withdrawn from all anti-anginal medications for two days prior to routine symptom-limited ETT (ACIP protocol). TWA was measured from lead V5 by Modified Moving Average analysis. Spatial T-wave heterogeneity (TWH) calculated by second central moment analysis of T-wave morphology from leads V4-6. TWA and TWH were measured at rest and at ETT heart rate of 120 beats/min, the peak achieved by all patients.

Participant Characteristics

Coronary artery disease patients (N=16) were adults of either sex chosen from a Vascular Basis Study of Ischemia, which required evidence of coronary artery disease in the form of greater than one millimeter (>1mm) ST-segment depression on ETT by the ACIP protocol and greater than one (>1) ischemic episode of greater than one minute (>1 min) on 48-hr AECG. In addition, Vascular Basis patients met criteria of greater than one (>1) native coronary obstruction of >50% luminal diameter, or myocardial infarction, or exercise-induced myocardial perfusion defect, or wall-motion abnormalities. Their total cholesterol was in the range of 180-250 mg/dl, with LDL >120 mg/dl while off lipid-lowering medication. Finally, they needed to be able to tolerate withdrawal from anti-ischemic medication for 72-96 hours. Patients were excluded from the Vascular Basis Study if they had recent unstable angina or acute MI, angioplasty or bypass, Congestive heart failure (NYHA III on medical therapy), Valvular disease, Diabetes with hemoglobin A1c>12%, uncontrolled hypertension (>160/100 mm Hg), or were on lipid lowering or antioxidant therapy, digoxin, or antidepressants known to produce ECG abnormalities, or were current smokers. They were also excluded if, at baseline, they exhibited ECG abnormalities that preclude accurate interpretation of ST-segment morphology including atrial fibrillation, pacemaker, left ventricular hypertrophy, resting ST-segment depression, LBBB, Q-waves in leads with reversible ST-segment deviation.

In the human clinical study, 75% (12/16) of the patients were hypertensive, 88% (14/16) were male; 50% (8/16) had a previous myocardial infarction, and their mean age was 64.8±2.2 years with an age range of 48 to 79 years.

The 16 normal volunteers had no known risk factors or cardiac medications and their ETT (ACIP-protocol) was negative for myocardial ischemia. As for the CAD patients, 88% (14/16) of the volunteers were male. Their mean age was 36.1±3.5 years, with an age range of 20 to 60 years.

Data Collection

During ETT (ACIP protocol), routine 12-lead ECGs were continuously recorded with standard electrodes. ECGs were digitized at 500 Hz per channel and stored on CD ROM. Preprocessing included reduction of high frequency noise, baseline wander and removal of ectopic and noisy beats.

T-Wave Heterogeneity Measurement

TWH was measured continuously by second central moment analysis across the JT interval of simultaneous beats from the standard precordial leads, as described herein above.

For inter-individual comparison, TWH values of all subjects were scaled to compensate for the overall reduction in ECG amplitude caused by poor contact or by low-amplitude signals received from diseased hearts. To address this problem, a scaling factor was developed that utilizes the height of the R-wave as a reference and involves multiplying the output TWH values for each individual by the inverse of the average QRS amplitude in microvolts. Specifically, in the human clinical study, the R-wave amplitudes of each of the N (e.g., three or four) leads used in the TWH calculation were averaged, and the TWH value was multiplied by 1000 μV/(average R-wave amplitude in microvolts).

Preprocessing of ECGs

Recording and analysis of data were performed with commercial equipment and preprocessed as described above for the animal experiments. Namely, ECGs were low-pass filtered at 50 Hz, sampled at 500 Hz per channel, and stored on rewritable optical disks by Streamer software. The data were down-sampled to 125 Hz for analysis on the MARS Workstation. Ectopic beats, ventricular arrhythmias, or artifacts automatically identified by the MARS workstation were verified by a trained operator and removed from analysis.

ECGs were low-pass filtered to remove high-frequency noise using an 8th order digital Butterworth filter with a comer frequency of 50 Hz. Baseline wander, a low-frequency artifact caused by changes in thoracic impedance during respiration, was estimated based on isoelectric points in each ECG beat by calculating a cubic spline and was subtracted from the ECG signal.

Results

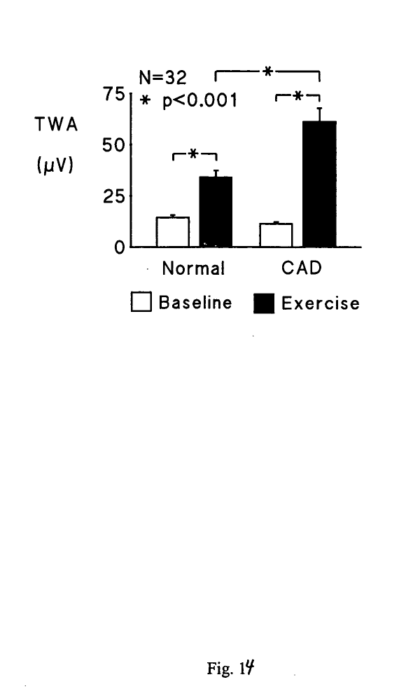

The TWA test results were interpretable in all cases, and baseline TWA and TWH did not differ appreciably between CAD patients and normals. FIG. 14 shows that, during ETT, TWA levels increased more in CAD patients than in normals (p<0.001). In normal subjects, TWA increased by 139% from baseline 14.46±1.15 to 34.54±2.95 μV at ETT heart rate of 120 beats/min (p<0.001). By contrast, in CAD patients, TWA increased by 444% from baseline 11.35±0.98 to 61.69±6.28 μV at ETT heart rate of 120 beats/min (p<0.001).

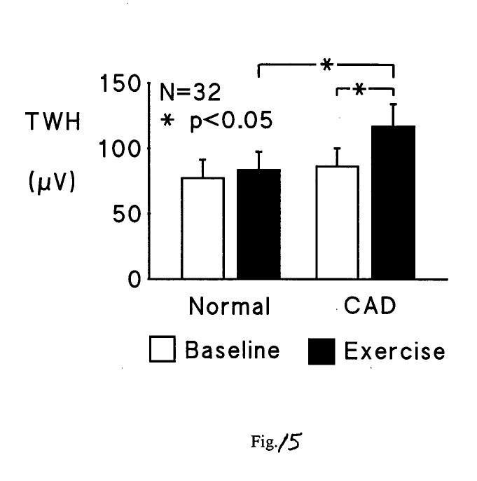

Simultaneously, TWH levels increased more in CAD patients than in normals during ETT (p<0.05). This is shown in FIG. 15. In normal subjects, TWH increased by 9% from baseline 77.29±14.14 to 84.21±13.46 μV at ETT heart rate of 120 beats/min (NS). By contrast, in CAD patients, TWH increased by 36% from baseline 86.42±13.80 to 117.73±16.51 μV at ETT heart rate of 120 beats/min (p<0.05). Thus, in CAD patients, exercise-induced TWA is associated with significant levels of spatial repolarization heterogeneity.

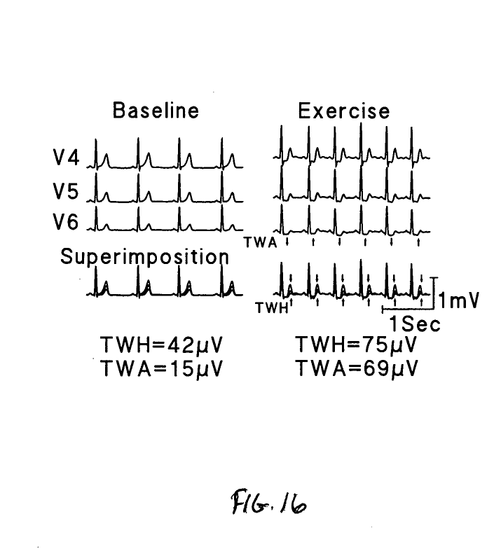

FIG. 16 shows a concurrent ETT-induced increase in TWA and TWH in a patient with coronary artery disease. Note that, prior to exercise, TWA is not visible in any of the three V leads, and only minor variance is visible in the morphology of the T-wave. During exercise, visible TWA is evident in lead V6, and TWH across the V leads is nearly double the resting value. TWA and TWH values are from lead V6.

Discussion of Human Clinical Study

The clinical studies focused on two objectives. First, to determine whether or not routine treadmill exercise elicits significant levels of TWA in patients with stable coronary artery disease. The second goal was to determine whether elevated levels of TWH occur concurrently, a finding that would provide a potential electrophysiologic basis for the observed TWA. To achieve accurate determinations of these electrophysiologic endpoints, we employed two newly developed signal-processing techniques, namely “modified moving average” analysis for TWA and “second central moment analysis” for TWH. Modified moving average analysis is described in U.S. Pat. No. 6,169,919 to Nearing and Verrier, which is incorporated herein by reference in its entirety as if reproduced in full below.

Exercise induced significantly greater levels of TWA in patients with stable coronary artery disease than in normal volunteers. There was a corresponding elevation in TWH, suggesting that heterogeneity of repolarization may underlie the increased TWA magnitude in patients with ischemic heart disease. TWH was not elevated in normal subjects.

Conclusions

The demonstration that there is a progressive increase in TWH that corresponds to the changes in TWA in CAD patients suggests that heterogeneity of repolarization may be an underlying mechanism accounting for exercise-induced TWA. Whereas the clinical study does not demonstrate a direct effect on susceptibility to VF, the observations in the porcine model using identical methodology for TWA and TWH assessment suggest plausibility of the hypothesis that the enhanced susceptibility to arrhythmias as a result of heterogeneity of repolarization may underlie the present clinical observations. In the human subjects, the ischemic events were less severe, and thus the actual occurrence of VF was not anticipated.

Extensive clinical studies indicate that maintaining exercise heart rates in the range of 110 to 120 beats/min provides optimum means to employ TWA to identify individuals at risk for arrhythmic events. The present results appear to be consistent with this guideline. Specifically, we determined for both TWA and TWH that a significant discrimination between patients with stable coronary disease and normal volunteers occurred in this range. An important feature of the present invention is, however, that stationarity of heart rate is not required. The techniques employed for both TWA and TWH are nonspectral and therefore do not require that heart rate remain fixed during testing. Signals are continuously acquired and determinations of both parameters output at, for example, 15-sec intervals.

The precise physiologic factors accounting for the change in TWA and TWH during exercise in CAD patients is unknown. It is likely, however, that both enhanced catecholamine levels and myocardial ischemia play a role, as these factors are prominent in exercising subjects with heart disease. Laboratory studies have shown that stimulation of sympathetic nerves or infusion of catecholamines both increase TWA levels and augment dispersion of repolarization. The present findings are also in agreement with the inventors' previous experimental studies of imposition of behavioral stress and clinical investigations in patients with ICDs who were challenged by mental arithmetic elicit, both of which elicit significant levels of TWA.

The present approach for assessing TWA and TWH offers distinct advantages over contemporary methods. Because it is nonspectral, it circumvents the need for fixation of heart rate and allows use of routine symptom-limited exercise protocols. The techniques can also be employed in the context of ambulatory ECG monitoring, as has been demonstrated for TWA in post-MI patients. No specialized electrodes are required, as accuracy of measurement is achieved by building on the power of signal averaging of numerous template complexes. Both measurements provide information about trends in the continuum of vulnerability.

TWA and TWH provide complementary means to assess cardiac electrical instability. Essentially, TWA is a measure of temporal inhomogeneities monitored from a single lead. TWH provides a spatial measure of heterogeneity, as signals from multiple leads are compared on a beat-by-beat basis. There is a complementary benefit that related physiologic phenomena are evaluated. Because these measures rely on different principles of determination, the potential for artifact in one may be offset by measurement principles of the other. For example, respiratory and rhythmic motion artifacts can disrupt the measurement of TWA, but, because TWH is a spatial approach, it is not disrupted by these potential artifacts.

In the experimental studies using the porcine model, there was a statistically significant correlation (r=0.86, p<0.0001) between TWA and TWH in the precordial leads with ischemia-induced VF. Thus, there appears to be a close linkage between TWA and TWH and vulnerability to VF, underscoring the potential of these parameters to provide indices of susceptibility to VF.

The findings of the studies described herein support the concept that myocardial ischemia-induced TWA during exercise is fundamentally linked to heterogeneity of repolarization, which may underlie the predictive utility of TWA in estimating risk of sudden cardiac death. The consistent provocation of TWA and TWH suggest that cardiac electrical instability may be assessed during routine ETT testing to evaluate clinical status. Moreover, combined assessment of temporal (TWA) and spatial (TWH) heterogeneity of repolarization may improve predictive accuracy and provide complementary insights into electrophysiologic mechanisms

Heightened levels of spatial heterogeneity of repolarization as assessed by second central moment analysis appear to underlie the progression from elevated TWA levels to more complex patterns and increased risk for VF. Detection of TWH could prove useful in elucidating mechanisms of VF. TWH monitored in precordial leads could contribute to stratifying risk for life-threatening arrhythmias.

The present method of quantification of TWH could be incorporated into diagnostic equipment to assist in the assessment of risk of sudden cardiac death. Furthermore, the method could be used to analyze data from an ambulatory ECG monitor (e.g., a Holter monitor) for the same purpose. In addition, appropriate hardware and/or software could be incorporated into an implantable cardioverter defibrillator (ICD) to implement the method of quantifying TWH. With an ICD implementation, upon detection of a predetermined level of TWH, appropriate therapy could be delivered to prevent VF and thereby avoid the necessity of delivering defibrillation shocks to a patient. Appropriate therapy could include, for example, electrical therapy (e.g., low energy anti-tachycardia pacing), drug therapy, and/or alerting the patient and/or physician of need to address the problem. In the case of drug therapy, the ICD could control an implanted drug infusion device that would deliver to the heart a sufficient quantity of an appropriate agent (e.g., a beta adrenergic or calcium channel blocker) to prevent VF. This yields a preemptive therapy for VF that is currently unavailable.

Furthermore, the invention may be used in a clinical setting with treadmill testing or ambulatory ECG monitoring to stratify risk for arrhythmia and to confirm diagnosis of myocardial ischemia, which can be unclear if only ST-segment depression or elevation is measured.

The invention also has application to cardiac resynchronization therapy (CRT) devices to identify potential arrhythmogenic affects requiring therapy or reprogramming of the device. In electrophysiologic (EP) laboratory applications, the invention may help to identify arrhythmogenic conditions and help to guide therapy.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

1. A method for assessing spatial heterogeneity of repolarization of a heart of a patient, comprising:
simultaneously sensing an ECG signal from each of a plurality of spatially separated leads attached to the patient;
for a plurality of N beats in each of the ECG signals, identifying a JT interval of each beat; and
for corresponding ones of the JT intervals of the ECG signals, calculating a second central moment indicative of spatial heterogeneity of repolarization.

2. The method of claim 1, wherein said calculating step comprises:
using temporally corresponding JT intervals of the N beats across the ECG signals to calculate an average amplitude across the leads for each JT interval; and
calculating, for each of the N beats of a selected one of the ECG signals, a difference in amplitude from the average amplitude for each JT interval.

3. The method of claim 1, wherein said calculating step comprises:
calculating for each JT interval an average JT interval amplitude across the plurality of leads;
calculating, for each JT interval of each ECG signal, a square of the difference between the corresponding average JT interval amplitude and the JT interval amplitude of the respective ECG signal;
calculating an average of the squares across the plurality of leads for each JT interval; and
calculating a square root of the average for each JT interval to produce for each JT interval a time-varying measure of spatial heterogeneity of repolarization.

4. The method of claim 3, further comprising:
computing a maximum amplitude value for each JT interval.

5. The method of claim 4, further comprising:
averaging the maximum amplitude values from the plurality of JT intervals.

6. The method of claim 3, further comprising:
computing an average amplitude value for each JT interval.

7. The method of claim 6, further comprising:
averaging the average amplitude values from a plurality of JT intervals.

8. A method for assessing spatial heterogeneity of repolarization of a heart of a patient, comprising:
(a) simultaneously sensing, from each of a plurality of spatially separated leads attached to the patient, an ECG signal having a plurality of N beats;
(b) using temporally corresponding JT intervals of the N beats across the ECG signals to calculate an average amplitude across the leads for each sample point in the JT intervals; and
(c) calculating, for each of the N beats of a selected one of the ECG signals, a magnitude difference in amplitude from the average amplitude for each sample point in the JT interval;
(d) repeating the calculating step for each ECG signal; and
(e) averaging the magnitude differences calculated in steps (c) and (d) to produce a measure of spatial heterogeneity of repolarization for each JT interval.

9. The method of claim 8, wherein each JT interval is represented by a plurality of samples, and wherein steps (b) through (e) operate on the plurality of samples.

10. The method of claim 8, wherein step (c) comprises calculating, for each of the samples of each of the N beats, a root mean square difference between the average JT interval amplitude and the JT interval of the selected ECG signal.

11. The method of claim 8, further comprising, before the using step, steps of:
comparing an amplitude of an R-wave of one of the ECG signal to a predetermined value; and
amplitude scaling the measure of heterogeneity when the comparing step so indicates.

12. The method of claim 8, further comprising:
computing a maximum amplitude value for each JT interval for the measure of spatial heterogeneity.

13. The method of claim 12, further comprising:
averaging the maximum amplitude values from the plurality of JT intervals.

14. An apparatus for assessing spatial heterogeneity of repolarization of a heart of a patient, comprising:
means for simultaneously sensing an ECG signal from each of a plurality of spatially separated leads attached to the patient;
means for identifying, for a plurality of N beats in each of the ECG signals, a JT interval of each beat; and
means for calculating, for corresponding ones of the JT intervals of the ECG signals, a second central moment indicative of spatial heterogeneity of repolarization.

15. The apparatus of claim 14, wherein said calculating means comprises:
means for using temporally corresponding JT intervals of the N beats across the ECG signals to calculate an average amplitude across the leads for each JT interval; and
means for calculating, for each of the N beats of a selected one of the ECG signals, a difference in amplitude from the average amplitude for each JT interval.

16. The apparatus of claim 14, wherein said calculating means comprises:
means for calculating for each JT interval an average JT interval amplitude across the plurality of leads;
means for calculating, for each JT interval of each ECG signal, a square of the difference between the corresponding average JT interval amplitude and the JT interval amplitude of the respective ECG signal;
means calculating an average of the squares across the plurality of leads for each JT interval; and
means for calculating a square root of the average for each JT interval to produce for each JT interval a time-varying measure of spatial heterogeneity of repolarization.

17. The apparatus of claim 16, further comprising:
means for computing a maximum amplitude value for each JT interval.

18. The apparatus of claim 16, further comprising:
means for averaging the maximum amplitude values from the plurality of JT intervals.

19. A method for sudden cardiac death risk identification and screening, comprising:
determining a measure of T wave heterogeneity for a patient;
scaling the measure based on a desired R-wave amplitude; and
comparing the measure to a normative value.

20. An apparatus for sudden cardiac death risk identification and screening, comprising:
means for determining a measure of T wave heterogeneity for a patient;
means for scaling the measure based on a desired R-wave amplitude; and
means for comparing the measure to a normative value.
